SEQUENCE LISTING (69 sequences)

SEQ ID NO: 1 - protein, 62 aa, Homo sapiens
LTPRRVTEVRADTGGLGRSITSPTTLYDRYSSPPASTTRRRLFVENDSPSDGGTPGRMPPQP

SEQ ID NO: 2 - protein, 42 aa, Homo sapiens
LTPRRVTEVRADTGGLGRSITSPTTLYDRYSSPPASTTRRRL

SEQ ID NO: 3 - protein, 39 aa, Homo sapiens
LTPRRVTEVRADTGGLGRSITSPTTLYDRYSSPPASTTR

SEQ ID NO: 4 - protein, 124 aa, Homo sapiens
VPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRL

SEQ ID NO: 5 - protein, 131 aa, Homo sapiens
LTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQ

SEQ ID NO: 6 - protein, 67 aa, Homo sapiens
VPTCEEVMPPQNLERADEICIAGSPLTPRRVTEVRADTGGLGRSITSPTTLYDRYSSPPASTTRRRL

SEQ ID NO: 7 - protein, 144 aa, Homo sapiens
VPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQP

SEQ ID NO: 8 - protein, 87 aa, Homo sapiens
VPTCEEVMPPQNLERADEICIAGSPLTPRRVTEVRADTGGLGRSITSPTTLYDRYSSPPASTTRRRLFVENDSPSDGGTPGRMPPQP

SEQ ID NO: 9 - protein, 121 aa, Homo sapiens
VPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTR

SEQ ID NO: 10 - protein, 64 aa, Homo sapiens
VPTCEEVMPPQNLERADEICIAGSPLTPRRVTEVRADTGGLGRSITSPTTLYDRYSSPPASTTR

SEQ ID NO: 11 - protein, 34 aa, Homo sapiens
LTPRRVTEVRADTGGLGRSITSPTTLYDRYSSPP

SEQ ID NO: 12 - protein, 35 aa, Homo sapiens
LTPRRVTEVRADTGGLGRSITSPTTLYDRYSSPPA

SEQ ID NO: 13 - protein, 36 aa, Homo sapiens
LTPRRVTEVRADTGGLGRSITSPTTLYDRYSSPPAS

SEQ ID NO: 14 - protein, 37 aa, Homo sapiens
LTPRRVTEVRADTGGLGRSITSPTTLYDRYSSPPAST

SEQ ID NO: 15 - protein, 38 aa, Homo sapiens
LTPRRVTEVRADTGGLGRSITSPTTLYDRYSSPPASTT

SEQ ID NO: 16 - protein, 10 aa, Artificial Sequence
  Description of Artificial Sequence Synthetic peptide
GRKKRRQRRR

SEQ ID NO: 17 - protein, 51 aa, Artificial Sequence
  Description of Artificial Sequence Synthetic peptide
GRKKRRQRRRPPLTPRRVTEVRADTGGLGRSITSPTTLYDRYSSPPASTTR

SEQ ID NO: 18 - protein, 51 aa, Artificial Sequence
  Description of Artificial Sequence Synthetic peptide
GRKKRRQRRRPPTDYSPATRSVGITRPTLPTSRDSYTGRERSVGPLRTALT

SEQ ID NO: 19 - protein, 39 aa, Artificial Sequence
  Description of Artificial Sequence Synthetic peptide
TDYSPATRSVGITRPTLPTSRDSYTGRERSVGPLRTALT

SEQ ID NO: 20 - protein, 213 aa, Homo sapiens
VPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSN

SEQ ID NO: 21 - protein, 1139 aa, Homo sapiens
MPSGGDQSPPPPPPPPAAAASDEEEEDDGEAEDAAPPAESPTPQIQQRFDELCSRLNMDEAARAEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIGTLSRCLNAGSGTETAERVQMKNILQQHFDKSKALRISTPLTGVRYIKENSPCVTPVSTATHSLSRLHTMLTGLRNAPSEKLEQILRTCSRDPTQAIANRLKEMFEIYSQHFQPDEDFSNCAKEIASKHFRFAEMLYYKVLESVIEQEQKRLGDMDLSGILEQDAFHRSLLACCLEVVTFSYKPPGNFPFITEIFDVPLYHFYKVIEVFIRAEDGLCREVVKHLNQIEEQILDHLAWKPESPLWEKIRDNENRVPTCEEVMPPQNLERADEICIAGSPLTPRRVTEVRADTGGLGRSITSPTTLYDRYSSPPASTTRRRLFVENDSPSDGGTPGRMPPQPLVNAVPVQNVSGETVSVTPVPGQTLVTMATATVTANNGQTVTIPVQGIANENGGITFFPVQVNVGGQAQAVTGSIQPLSAQALAGSLSSQQVTGTTLQVPGQVAIQQISPGGQQQKQGQSVTSSSNRPRKTSSLSLFFRKVYHLAAVRLRDLCAKLDISDELRKKIWTCFEFSIIQCPELMMDRHLDQLLMCAIYVMAKVTKEDKSFQNIMRCYRTQPQARSQVYRSVLIKGKRKRRNSGSSDSRSHQNSPTELNKDRTSRDSSPVMRSSSTLPVPQPSSAPPTPTRLTGANSDMEEEERGDLIQFYNNIYIKQIKTFAMKYSQANMDAPPLSPYPFVRTGSPRRIQLSQNHPVYISPHKNETMLSPREKIFYYFSNSPSKRLREINSMIRTGETPTKKRGILLEDGSESPAKRICPENHSALLRRLQDVANDRGSH

SEQ ID NO: 22 - protein, 9 aa, Homo sapiens
LTPRRVTEV

SEQ ID NO: 23 - protein, 11 aa, Homo sapiens
ASTTRRRLFVE

SEQ ID NO: 24 - protein, 14 aa, Homo sapiens
TLYDRYSSPPASTT

SEQ ID NO: 25 - protein, 25 aa, Homo sapiens
GLGRSITSPTTLYDRYSSPPASTTR

SEQ ID NO: 26 - protein, 30 aa, Homo sapiens
RADTGGLGRSITSPTTLYDRYSSPPASTTR

SEQ ID NO: 27 - protein, 36 aa, Homo sapiens
RRVTEVRADTGGLGRSITSPTTLYDRYSSPPASTTR

SEQ ID NO: 28 - protein, 14 aa, Homo sapiens
LTPRRVTEVRADTG

SEQ ID NO: 29 - protein, 19 aa, Homo sapiens
LTPRRVTEVRADTGGLGRS

SEQ ID NO: 30 - protein, 24 aa, Homo sapiens
LTPRRVTEVRADTGGLGRSITSPT

SEQ ID NO: 31 - protein, 29 aa, Homo sapiens
LTPRRVTEVRADTGGLGRSITSPTTLYDR

SEQ ID NO: 32 - protein, 34 aa, Homo sapiens; identical to SEQ ID NO: 11

SEQ ID NO: 33 - protein, 20 aa, Homo sapiens
ITSPTTLYDRYSSPPASTTR

SEQ ID NO: 34 - protein, 9 aa, Homo sapiens
LTPRRVTEV

SEQ ID NO: 35 - protein, 39 aa, Homo sapiens
LTPARVTEVRADTGGLGRSITSPTTLYDRYSSPPASTTR

SEQ ID NO: 36 - protein, 39 aa, Homo sapiens
LTPRRATEVRADTGGLGRSITSPTTLYDRYSSPPASTTR

SEQ ID NO: 37 - protein, 39 aa, Homo sapiens
LTPARATEVRADTGGLGRSITSPTTLYDRYSSPPASTTR

SEQ ID NO: 38 - protein, 39 aa, Homo sapiens
LTPRRVTEVAADTGGLGRSITSPTTLYDRYSSPPASTTR

SEQ ID NO: 39 - protein, 38 aa, Homo sapiens
TPRRVTEVRADTGGLGRSITSPTTLYDRYSSPPASTTR

SEQ ID NO: 40 - protein, 37 aa, Homo sapiens
PRRVTEVRADTGGLGRSITSPTTLYDRYSSPPASTTR

SEQ ID NO: 41 - protein, 35 aa, Homo sapiens
RVTEVRADTGGLGRSITSPTTLYDRYSSPPASTTR

SEQ ID NO: 42 - protein, 34 aa, Homo sapiens
VTEVRADTGGLGRSITSPTTLYDRYSSPPASTTR

SEQ ID NO: 43 - protein, 33 aa, Homo sapiens
TEVRADTGGLGRSITSPTTLYDRYSSPPASTTR

SEQ ID NO: 44 - protein, 37 aa, Homo sapiens
TPRRVTEVRADTGGLGRSITSPTTLYDRYSSPPASTT

SEQ ID NO: 45 - protein, 36 aa, Homo sapiens
TPRRVTEVRADTGGLGRSITSPTTLYDRYSSPPAST

SEQ ID NO: 46 - protein, 35 aa, Homo sapiens
TPRRVTEVRADTGGLGRSITSPTTLYDRYSSPPAS

SEQ ID NO: 47 - protein, 34 aa, Homo sapiens
TPRRVTEVRADTGGLGRSITSPTTLYDRYSSPPA

SEQ ID NO: 48 - protein, 33 aa, Homo sapiens
TPRRVTEVRADTGGLGRSITSPTTLYDRYSSPP

SEQ ID NO: 49 - protein, 36 aa, Homo sapiens
PRRVTEVRADTGGLGRSITSPTTLYDRYSSPPASTT

SEQ ID NO: 50 - protein, 35 aa, Homo sapiens
PRRVTEVRADTGGLGRSITSPTTLYDRYSSPPAST

SEQ ID NO: 51 - protein, 34 aa, Homo sapiens
PRRVTEVRADTGGLGRSITSPTTLYDRYSSPPAS

SEQ ID NO: 52 - protein, 33 aa, Homo sapiens
PRRVTEVRADTGGLGRSITSPTTLYDRYSSPPA

SEQ ID NO: 53 - protein, 32 aa, Homo sapiens
PRRVTEVRADTGGLGRSITSPTTLYDRYSSPP

SEQ ID NO: 54 - protein, 34 aa, Homo sapiens
RVTEVRADTGGLGRSITSPTTLYDRYSSPPASTT

SEQ ID NO: 55 - protein, 33 aa, Homo sapiens
RVTEVRADTGGLGRSITSPTTLYDRYSSPPAST

SEQ ID NO: 56 - protein, 32 aa, Homo sapiens
RVTEVRADTGGLGRSITSPTTLYDRYSSPPAS

SEQ ID NO: 57 - protein, 31 aa, Homo sapiens
RVTEVRADTGGLGRSITSPTTLYDRYSSPPA

SEQ ID NO: 58 - protein, 30 aa, Homo sapiens
RVTEVRADTGGLGRSITSPTTLYDRYSSPP

SEQ ID NO: 59 - protein, 33 aa, Homo sapiens
VTEVRADTGGLGRSITSPTTLYDRYSSPPASTT

SEQ ID NO: 60 - protein, 32 aa, Homo sapiens
VTEVRADTGGLGRSITSPTTLYDRYSSPPAST

SEQ ID NO: 61 - protein, 31 aa, Homo sapiens
VTEVRADTGGLGRSITSPTTLYDRYSSPPAS

SEQ ID NO: 62 - protein, 30 aa, Homo sapiens
VTEVRADTGGLGRSITSPTTLYDRYSSPPA

SEQ ID NO: 63 - protein, 29 aa, Homo sapiens
VTEVRADTGGLGRSITSPTTLYDRYSSPP

SEQ ID NO: 64 - protein, 32 aa, Homo sapiens
TEVRADTGGLGRSITSPTTLYDRYSSPPASTT

SEQ ID NO: 65 - protein, 31 aa, Homo sapiens
TEVRADTGGLGRSITSPTTLYDRYSSPPAST

SEQ ID NO: 66 - protein, 30 aa, Homo sapiens
TEVRADTGGLGRSITSPTTLYDRYSSPPAS

SEQ ID NO: 67 - protein, 29 aa, Homo sapiens
TEVRADTGGLGRSITSPTTLYDRYSSPPA

SEQ ID NO: 68 - protein, 28 aa, Homo sapiens
TEVRADTGGLGRSITSPTTLYDRYSSPP

SEQ ID NO: 69 - protein, 11 aa, Homo sapiens
ASTTRRRLFVE

(19) United States
(12) Patent Application Publication
(10) Pub. No.: US 2009/0054333 A1
(43) Pub. Date: Feb. 26, 2009

(54) Peptide inhibitors of cyclin-dependent kinase activity and uses thereof

(76) Inventor: Antonio Giordano, Radnor, PA (US)

Correspondence Address: REED SMITH LLP, 2500 ONE LIBERTY PLACE, 1650 MARKET STREET, PHILADELPHIA, PA, 19103, US

(21) Appl. No.: 12/148,282

(22) Filed: Apr. 16, 2008

Related U.S. Application Data
(63) Continuation-in-part of application No. 11/582,871, filed on Oct. 17, 2006, now Pat. No. 7,470,670.

(51) Int. Cl. C07K 7/06; C07K 7/08; C07K 14/00; A61K 38/16; A61K 38/10; A61K 38/08; A61P 35/04

(52) U.S. Cl. 514/12; 530/328; 530/327; 530/326; 530/325; 530/324; 514/13; 514/14; 514/15

(57) ABSTRACT

Novel polypeptides or derivatives comprising cdk2 binding site are disclosed. The novel polypeptides or derivatives have growth suppressive activity. Nucleic acids encoding those polypeptides are also disclosed. The polypeptides identified herein are also useful in methods for treating or preventing cancer. The treatment methods comprise administration of the polypeptide to the patient. The methods also comprise contacting the sample with the above-described polypeptide or derivative, wherein the polypeptide or derivative also comprises a covalently attached detectable moiety, then determining whether the polypeptide or derivative is binding cdk2 from the sample.

### CROSS-REFERENCE TO RELATED APPLICATIONS

This application claims priority to U.S. patent application Ser. No. 11/582,871, which was filed on Oct. 17, 2006, which is incorporated herein by reference in its entirety including all references cited therein.

### STATEMENT REGARDING FEDERALLY SPONSORED RESEARCH

The subject matter of this application was made possible, in part, with funding from the U.S. Government. The Government may have certain rights.

### TECHNICAL FIELD OF THE INVENTION

Generally, the present invention relates to inhibitors of cyclin-dependent kinase activity and, more particularly, pharmaceutical compositions containing the compounds, and the use of the compounds for the treatment of cancer and tumors. The present invention especially provides isolated and specific active components of the pRb2/p130 gene responsible for growth suppressive activity.

### BACKGROUND OF THE INVENTION

One of the main goals in the development of novel therapeutics for proliferative disorders is to generate selective small molecules that potently inhibit cell cycle progression. Several studies have provided evidence of the critical involvement of cyclin/cdk complexes at specific cell cycle regulatory checkpoints (Morgan, 1997, Annu Rev Cell Dev Biol, 13, 261-91; Sherr, 1996, Science, 274, 1672-7). Progression through the cell cycle is driven by activation and deactivation of cyclin/cdk complexes, which start a fundamental cascade of events leading to DNA replication and chromosomal segregation. Tumor development is closely associated with alteration and deregulation of cdks and their regulators, suggesting that inhibitors (antagonists) of cdks may be useful anticancer therapeutics. Therefore, targeting cdk activity has become an attractive strategy in cancer therapy, since it could potentially create a rationally designed inhibitor of a specific process that leads a cell to malignant transformation. To date, several families of chemical inhibitors targeted against different cdk activities have been described (Gray et al., 1998, Science, 281, 533-8; Losiewicz et al., 1994, Biochem Biophys Res Commun, 201, 589-95) and, for some of them, their anticancer therapeutic potential has been demonstrated in preclinical studies (Dai & Grant, 2004, Curr Oncol Rep, 6, 123-30). Recent attention has been focused on biological molecules, especially peptide antagonists, rather than chemotherapeutic agents, that combine the effectiveness of arresting cellular growth through interaction with important cell cycle checkpoint regulators and the low risk of unexpected adverse reactions, thus improving clinical safety and patient tolerability. Therefore, development of pharmacological small peptide molecules able to inhibit cdk activity could be an alternative mechanism-based therapy of great interest in the treatment of neoplasms or other proliferative disorders.

Cdk2 is known to be active in complex with cyclin E at the G1-S boundary, and in complex with cyclin A during S phase progression (Sherr, 1996, Science, 274, 1672-7). Cyclin-dependent kinase 2 (cdk2) is considered the prototypic cell cycle kinase and plays a crucial role in the regulation of cell cycle progression in mammalian cells (Koff et al., 1992, Science, 257, 1689-94; Ohtsubo et al., 1995, Mol Cell Biol, 15, 2612-24). Cdk2 is necessary to pass the G1 restriction point and to drive cells into DNA replication. This enzyme determines whether a cell will leave its resting phase and enter the S phase, a critical determining point, after which a cell is committed to divide.

Among the target substrates that cdks phosphorylate are the members of the retinoblastoma (Rb) family proteins, which play a pivotal role as negative regulators of cell cycle progression (Claudio et al., 1994, Cancer Res, 54, 5556-60). This family includes the product of the retinoblastoma susceptibility gene, the pRb/p105 protein, and the related p107 and pRb2/130 proteins (Hannon et al., 1993, Genes Dev, 7, 2378-91; Mayol et al., 1993, Oncogene, 8, 2561-6; Paggi et al., 1996, J Cell Biochem, 62, 418-30). They share the ability to recruit chromatin-remodeling enzymes and their best characterized targets are the members of the E2F/DP family of transcription factors, generally referred to as E2F (Weinberg, 1995, Cell, 81, 323-30). Both pRb2/p130 and p107 are able to bind cdk2/cyclins A and E (Claudio et al., 1996, Cancer Res, 56, 2003-8). Overexpression of cdk2 with associated cyclins has been shown in several tumors (Al-Aynati et al., 2004, Clin Cancer Res, 10, 6598-605; Olofsson et al., 2004, Int J. Oncol, 25, 1349-55; Zhu, 2004, Cell Cycle, 3). Furthermore, cdk2 has been recently found to be required for centrosome duplication in mammalian cells (Matsumoto et al., 1999, Curr Biol, 9, 429-32; Matsumoto & Maller, 2004, Science, 306, 885-8) suggesting that inhibition of cdk2 activity would be an effective anti-cancer approach. In addition, cdk2 has rapidly emerged as a potential inhibition target by small molecule drugs, which should eventually lead to the development of effective therapies for proliferative disorders (Andrews et al., 2004, Org Biomol Chem, 2, 2735-41; Dai & Grant, 2004, Curr Oncol Rep, 6, 123-30; Gibbs & Oliff, 1994, Cell, 79, 193-8; Hsu et al., 2004, Life Sci, 75, 2303-16; Senderowicz, 2003, Oncogene, 22, 6609-20; Song et al., 2004, Biochem Biophys Res Commun, 317, 128-32).

Previously, it was demonstrated that pRb2/p130, a member of the retinoblastoma family of proteins, acts during cell growth suppression as an inhibitor of cdk2 activity (De Luca et al., 1997, J Biol Chem, 272, 20971-4). The spacer region of pRb2/p130 has a unique amino acid sequence among the other members of the retinoblastoma family, and it is responsible for this inhibitory effect on cdk2 (U.S. Pat. No. 6,297,357). U.S. Pat. Nos. 5,457,049; 5,532,340; 5,807,681; 5,840,506; and 6,663,856, each of which is herein incorporated by reference in its entirety including any references cited therein, also disclose the nucleic acid and polypeptide sequences of the pRB2/p130 spacer domain. The identification and isolation of further true cdk inhibitor peptides exhibiting growth suppressive activity would be useful for designing treatments for cancer therapy; either as an alternative to or in conjunction with other known therapies.

### SUMMARY OF THE INVENTION

The invention relates to the discovery of pharmacological polypeptide molecules that are able to inhibit cell cycle progression and induce growth arrest when expressed in cells and promote tumor regression in vivo. The polypeptide molecules disclosed in the present invention contain a fragment of the full-length sequence of the pRb2/p130 spacer domain (amino acids 616-828) (SEQ ID NO:20). The peptide molecules are specific to and capable of inhibiting cdk2-dependent histone phosphorylation and halting cellular growth by arresting cells in the G₀/G₁ phase of the cell cycle.

Accordingly, in one aspect, the invention provides cdk2 kinase activity inhibiting peptides and nucleic acid fragments of pRb2/p130 encoding the polypeptides, referred to herein as Spa peptide molecules (Spa polypeptides/Spa peptides) and Spa nucleic acid molecules (Spa nucleic acid), respectively.

The Spa peptide molecules contain contiguous amino acids of between about 34 to about 144 amino acids long and contain at least amino acids 641 to 674 of the spacer domain wherein the Spa peptide molecules are capable of inhibiting cdk2 kinase activity; and are about 39 to 70 amino acids in length, beginning with amino acid 641 and including amino acid 674 of the spacer domain.

The invention provides at least 10 different Spa peptide molecules as well as functionally equivalent subsets of these molecules, including but not limited to deletion mutants and variants thereof. For purposes of the present invention, variants are only those that carry amino acid substitutions in the fragments from the spacer molecule. The deletion mutants and variants are collectively referred to herein as “mutants,” include only those determined to have cdk2 inhibitory activity.

According to an embodiment, the invention provides a cdk2 activity inhibiting polypeptide having one of the following amino acid sequences based upon the native amino acid sequence of pRb2/p130 (SEQ ID NO:21): amino acids 641 to 702 (62 amino acids long) designated Spa38 (SEQ ID NO:1); amino acids 641 to 682 (42 amino acids long) designated Spa311 (SEQ ID NO:2); amino acids 641-679 (39 amino acids long) designated Spa310 (SEQ ID NO:3); amino acids 559 to 682 (124 amino acids long) designated Spa319 (SEQ ID NO:4); amino acids 641-771 (131 amino acids long) designated Spa313 (SEQ ID NO:5); amino acids 616-682 (67 amino acids long) designated Spa314 (SEQ ID NO:6); amino acids 559-702 (144 amino acids long) designated Spa315 (SEQ ID NO:7); amino acids 616 to 702 (87 amino acids long) designated Spa316 (SEQ ID NO:8); amino acids 559-679 (121 amino acids long) designated Spa317 (SEQ ID NO:9); amino acids 616 to 679 (64 amino acids long) designated Spa318 (SEQ ID NO:10); amino acids 641 to 674 (34 amino acids long) designated Spa20 (SEQ ID NO:11); amino acids 641 to 675 (35 amino acids long) designated Spa21 (SEQ ID NO:12); amino acids 641 to 676 (36 amino acids long) designated Spa22 (SEQ ID NO:13); amino acids 641 to 677 (37 amino acids long) designated Spa23 (SEQ ID NO:14); and amino acids 641 to 678 (38 amino acids long) designated Spa24 (SEQ ID NO: 15) or the fragments/deletion mutants of any of SEQ ID NOs: 1-15.

According to another preferred embodiment, the Spa peptide molecules of the present invention are no more than 39 amino acid long polypeptides, contain at least 9 contiguous amino acids of Spa310 (amino acids 641-679) (SEQ ID NO:3), and are capable of inhibiting cdk2 kinase activity. The invention includes compounds having a cdk2 activity inhibiting polypeptide having one of the following amino acid sequences based upon the native amino acid sequence of pRb2/p130 (SEQ ID NO:21): amino acids 641 to 649 (9 amino acids long) designated Spa40 (SEQ ID NO:22); amino acids 675 to 685 (11 amino acids long) designated Spa41 (SEQ ID NO:23); amino acids 665 to 678 (15 amino acids long) designated Spa42 (SEQ ID NO:24); amino acids 655 to 679 (25 amino acids long) designated Spa43 (SEQ ID NO:25); amino acids 650 to 679 (30 amino acids long) designated Spa44 (SEQ ID NO:26); amino acids 644 to 679 (36 amino acids long) designated Spa45 (SEQ ID NO:27); amino acids 641 to 654 (14 amino acids long) designated Spa46 (SEQ ID NO:28); amino acids 641 to 659 (19 amino acids long) designated Spa47 (SEQ ID NO:29); amino acids 641 to 664 (24 amino acids long) designated Spa48 (SEQ ID NO:30); amino acids 641 to 669 (29 amino acids long) designated Spa49 (SEQ ID NO:31); amino acids 641 to 674 (34 amino acids long) designated Spa50 (SEQ ID NO:32); amino acids 660 to 679 (20 amino acids long) designated Spa51 (SEQ ID NO:33); amino acids 641 to 649 (9 amino acids long) designated Spa52 (SEQ ID NO:34); amino acids 641 to 679 (39 amino acids long) wherein amino acid 644 is alanine, designated Spa54 (SEQ ID NO: 35); amino acids 641 to 679 (39 amino acids long) wherein amino acid 646 is alanine, designated Spa55 (SEQ ID NO: 36); amino acids 641 to 679 (39 amino acids long) wherein amino acids 644 and 646 are alanine, designated Spa56 (SEQ ID NO:37); amino acids 641 to 679 (39 amino acids long) wherein amino acids 650 is alanine, designated Spa57 (SEQ ID NO:38); amino acids 642 to 679 (38 amino acids long) designated Spa58 (SEQ ID NO:39); amino acids 643 to 679 (37 amino acids long) designated Spa59 (SEQ ID NO:40); amino acids 645 to 679 (36 amino acids long) designated Spa60 (SEQ ID NO:41); amino acids 646 to 679 (35 amino acids long) designated Spa61 (SEQ ID NO:42); amino acids 647 to 679 (34 amino acids long) designated Spa62 (SEQ ID NO:43); amino acids 642 to 678 (37 amino acids long) designated Spa63 (SEQ ID NO:44); amino acids 642 to 677 (36 amino acids long) designated Spa64 (SEQ ID NO:45); amino acids 642 to 676 (35 amino acids long) designated Spa65 (SEQ ID NO:46); amino acids 642 to 675 (34 amino acids long) designated Spa66 (SEQ ID NO:47); amino acids 642 to 674 (33 amino acids long) designated Spa67 (SEQ ID NO:48); amino acids 643 to 678 (36 amino acids long) designated Spa68 (SEQ ID NO:49); amino acids 643 to 677 (35 amino acids long) designated Spa69 (SEQ ID NO:50); amino acids 643 to 676 (34 amino acids long) designated Spa70 (SEQ ID NO:51); amino acids 643 to 675 (33 amino acids long) designated Spa71 (SEQ ID NO:52); amino acids 643 to 674 (32 amino acids long) designated Spa72 (SEQ ID NO:53); amino acids 645 to 678 (34 amino acids long) designated Spa73 (SEQ ID NO:54); amino acids 645 to 677 (33 amino acids long) designated Spa74 (SEQ ID NO:55); amino acids 645 to 676 (32 amino acids long) designated Spa75 (SEQ ID NO:56); amino acids 645 to 675 (31 amino acids long) designated Spa76 (SEQ ID NO:57); amino acids 645 to 674 (30 amino acids long) designated Spa77 (SEQ ID NO:58); amino acids 646 to 678 (33 amino acids long) designated Spa78 (SEQ ID NO:59); amino acids 646 to 677 (32 amino acids long) designated Spa79 (SEQ ID NO:60); amino acids 646 to 676 (31 amino acids long) designated Spa80 (SEQ ID NO:61); amino acids 646 to 675 (30 amino acids long) designated Spa81 (SEQ ID NO:62); amino acids 646 to 674 (29 amino acids long) designated Spa82 (SEQ ID NO:63); amino acids 647 to 678 (32 amino acids long) designated Spa83 (SEQ ID NO:64); amino acids 647 to 677 (31 amino acids long) designated Spa84 (SEQ ID NO:65); amino acids 647 to 676 (30 amino acids long) designated Spa85 (SEQ ID NO:66); amino acids 647 to 675 (29 amino acids long) designated Spa86 (SEQ ID NO:67); amino acids 647 to 674 (28 amino acids long) designated Spa87 (SEQ ID NO:68); or the fragments/deletion mutants of any of SEQ ID NOs: 22-68.

According to an embodiment, the Spa peptide molecules of the present invention are between 11 and 38 amino acid long polypeptides, contain at least 5 contiguous amino acids of Spa30 (amino acids 641-679) (SEQ ID NO:3), and capable of inhibiting cdk2 kinase activity. The invention includes compounds having a cdk2 activity inhibiting polypeptide having the following amino acid sequence based upon the native amino acid sequence of pRb2/p130 (SEQ ID NO:21): amino acids 675 to 685 (11 amino acids long) designated Spa53 (SEQ ID NO:69); and fragments/deletion mutants thereof.

The variants can be different from the fragments of the spacer molecules described herein (e.g., Spa310 peptide, Spa311 peptide or Spa38 peptide) at one, two, three, four, or five amino acid positions. In the most common instances, such differences will involve conservative amino acid substitutions. In one embodiment, the amino acid sequence of a variant of the present invention is identical to that set forth in SEQ ID NOs: 1, 2 or 3 except that, over the entire length corresponding to the amino acid sequence of SEQ ID NOs: 1, 2 or 3, the variant amino acid sequence has one, two, three, four, or five amino acid substitutions, preferably, conservative amino acid substitutions.

Accordingly, for the purposes of the present invention, by “peptide” it is meant to include peptides having any of SEQ ID NOs: 1-15, SEQ ID NOs: 22-69, or fragments or mutants thereof having cdk2 inhibitory activity. These peptide sequences, which retain the inhibitory activity, can be prepared synthetically in accordance with well-known methods such as solid or solution phase peptide synthesis. Alternatively, peptides of the present invention may be synthesized recombinantly.

These and other aspects of the invention will be described in greater detail herein.

### BRIEF DESCRIPTION OF THE DRAWINGS

The present invention is best understood with reference to the following detailed description of the invention and the drawings in which:

FIG. 1 shows an embodiment of the invention where cdk2 was immunoprecipitated with anti-cdk2 polyclonal antibody from exponentially growing NIH/3T3 incubated with equal amounts of GST-fusion proteins in a kinase reaction mixture, and assessed for phosphorylation ability of the histone H1 substrate; and using SDS-PAGE gels (10%), various constructs of pRb2/p130 maintain the inhibitory effects of the pRB2 μl 30 spacer domain on cdk2 kinase activity;

FIG. 2 shows an embodiment of the invention of subcellular localization of the Spacer and Spa310 molecules by immunofluorescent staining;

FIGS. 3a and 3b show an embodiment of the invention using SDS-PAGE gels (10%) where Spa310 and the Spacer inhibit endogenous cdk2 activity;

FIGS. 4a and 4b illustrate an embodiment of the invention where Spa310 suppresses colony formation similarly to the full-length spacer domain of pRb2/p130;

FIGS. 5a and 5b illustrate an embodiment of the invention where the flow cytometry shows the effect of ectopic expression of pRb2/p130-spacer and Spa310 on the cell cycle; and

FIG. 6 is a graph of an embodiment of the invention showing tumor suppressive effects of four different peptides (TAT-Spa310, TAT-SCRAMBLE, Spa310 and SCRAMBLE) in tumor bearing mice.

### DETAILED DESCRIPTION

Reference will now be made in detail to embodiments of the invention, examples of which are illustrated in the accompanying drawings. While the invention will be described in conjunction with the embodiments, it will be understood that they are not intended to limit the invention to those embodiments. On the contrary, the invention is intended to cover alternatives, modifications, and equivalents, which may be included within the spirit and scope of the invention as defined by the appended claims.

The invention relates in part to the identification and use of peptides that specifically inhibit cdk2 activity. These peptides are referred to herein as Spa peptide molecules. These peptide molecules are able to greatly inhibit cdk2-dependent histone phosphorylation and halt cellular growth by arresting cells in the G0/G1 phase of the cell cycle. The peptides and their functional equivalents are useful in the diagnosis and treatment of disorders characterized by cdk2 expression or overexpression. In particular, the peptides and their functional equivalents are useful in the treatment of neoplasms or other proliferative disorders including cancer and tumors, in particular for the prevention and inhibition of tumor colonization. They are also useful in the isolation and, optionally, removal of cells that overexpress cdk2 (e.g., tumor cells). These peptides can also be used to identify further Spa peptide molecules.

According to an embodiment, the invention involves, in various related and interconnected aspects, isolated cdk2-inhibiting peptides, functional equivalents and modifications and variants thereof, unique fragments thereof, nucleic acid molecules encoding the foregoing, as well as diagnostics and therapeutics relating thereto.

According to an embodiment, the invention includes a composition having a cdk2 activity inhibiting polypeptide having one of the following amino acid sequences based upon the native amino acid sequence of pRb2/p130 (SEQ ID NO:21): amino acids 641 to 702 (62 amino acids long) designated Spa38 (SEQ ID NO:1); amino acids 641 to 682 (42 amino acids long) designated Spa311 (SEQ ID NO:2); amino acids 641-679 (39 amino acids long) designated Spa310 (SEQ ID NO:3); amino acids 559 to 682 (124 amino acids long) designated Spa319 (SEQ ID NO:4); amino acids 641-771 (131 amino acids long) designated Spa313 (SEQ ID NO:5); amino acids 616-682 (67 amino acids long) designated Spa314 (SEQ ID NO:6); amino acids 559-702 (144 amino acids long) designated Spa315 (SEQ ID NO:7); amino acids 616 to 702 (87 amino acids long) designated Spa316 (SEQ ID NO:8); amino acids 559-679 (121 amino acids long) designated Spa317 (SEQ ID NO:9); amino acids 616 to 679 (64 amino acids long) designated Spa318 (SEQ ID NO:10); amino acids 641 to 674 (34 amino acids long) designated Spa20 (SEQ ID NO:11); amino acids 641 to 675 (35 amino acids long) designated Spa21 (SEQ ID NO:12); amino acids 641 to 676 (36 amino acids long) designated Spa22 (SEQ ID NO:13); amino acids 641 to 677 (37 amino acids long) designated Spa23 (SEQ ID NO:14); and amino acids 641 to 678 (38 amino acids long) designated Spa24 (SEQ ID NO: 15) or the fragments/deletion mutants of any of SEQ ID NOs: 1-15.

The variants can be different from the fragments of the spacer molecules described herein (e.g., Spa310 peptide, Spa311 peptide or Spa38 peptide) at one, two, three, four, or five amino acid positions. In the most common instances, such differences will involve conservative amino acid substitutions. In one embodiment, the amino acid sequence of a variant of the present invention is identical to that set forth in SEQ ID NOs: 1, 2 or 3 except that, over the entire length corresponding to the amino acid sequence of SEQ ID NOs: 1, 2 or 3, the variant amino acid sequence has one, two, three, four, or five amino acid substitutions, preferably, conservative amino acid substitutions. In some embodiments, variants are peptides that have at least 80%, at least 85%, at least 90%, or at least 95% identity (match) but not 100% identity over the full length of the Spa peptides described herein and determined to have cdk2 inhibitory activity. Conservative substitutions of amino acids include, but are not limited to substitutions made amongst amino acids within the following groups: (i) F, Y, W; (ii) K, R, H; (iii) M, I, L, V; and (iv) E, D.

According to a preferred embodiment, the Spa peptide molecules of the present invention are no more than 70 amino acids long polypeptides, contain at least amino acids 641-674, and are of sufficient length capable of inhibiting cdk2 kinase activity. A functionally equivalent variant of such a 70 amino acid long polypeptide is one that is identical to the 70 amino acid long polypeptide except that, over the entire length corresponding to the amino acid sequence of the 70 amino acid polypeptide, the variant has one, two, three, four, or five conservative amino acid substitutions.

According to another preferred embodiment, the Spa peptide molecules of the present invention are no more than 39 amino acid long polypeptides, contain at least 9 contiguous amino acids of Spa310 (amino acids 641-679) (SEQ ID NO:3), and are capable of inhibiting cdk2 kinase activity. The invention includes compounds having a cdk2 activity inhibiting polypeptide having one of the following amino acid sequences based upon the native amino acid sequence of pRb2/p130 (SEQ ID NO:21): amino acids 641 to 649 (9 amino acids long) designated Spa40 (SEQ ID NO:22); amino acids 675 to 685 (11 amino acids long) designated Spa41 (SEQ ID NO:23); amino acids 665 to 678 (15 amino acids long) designated Spa42 (SEQ ID NO:24); amino acids 655 to 679 (25 amino acids long) designated Spa43 (SEQ ID NO:25); amino acids 650 to 679 (30 amino acids long) designated Spa44 (SEQ ID NO:26); amino acids 644 to 679 (36 amino acids long) designated Spa45 (SEQ ID NO:27); amino acids 641 to 654 (14 amino acids long) designated Spa46 (SEQ ID NO:28); amino acids 641 to 659 (19 amino acids long) designated Spa47 (SEQ ID NO:29); amino acids 641 to 664 (24 amino acids long) designated Spa48 (SEQ ID NO:30); amino acids 641 to 669 (29 amino acids long) designated Spa49 (SEQ ID NO:31); amino acids 641 to 674 (34 amino acids long) designated Spa50 (SEQ ID NO:32); amino acids 660 to 679 (20 amino acids long) designated Spa51 (SEQ ID NO:33); amino acids 641 to 649 (9 amino acids long) designated Spa52 (SEQ ID NO:34); amino acids 641 to 679 (39 amino acids long) wherein amino acid 644 is alanine, designated Spa54 (SEQ ID NO: 35); amino acids 641 to 679 (39 amino acids long) wherein amino acid 646 is alanine, designated Spa55 (SEQ ID NO: 36); amino acids 641 to 679 (39 amino acids long) wherein amino acids 644 and 646 are alanine, designated Spa56 (SEQ ID NO:37); amino acids 641 to 679 (39 amino acids long) wherein amino acids 650 is alanine, designated Spa57 (SEQ ID NO:38); amino acids 642 to 679 (38 amino acids long) designated Spa58 (SEQ ID NO:39); amino acids 643 to 679 (37 amino acids long) designated Spa59 (SEQ ID NO:40); amino acids 645 to 679 (36 amino acids long) designated Spa60 (SEQ ID NO:41); amino acids 646 to 679 (35 amino acids long) designated Spa61 (SEQ ID NO:42); amino acids 647 to 679 (34 amino acids long) designated Spa62 (SEQ ID NO:43); amino acids 642 to 678 (37 amino acids long) designated Spa63 (SEQ ID NO:44); amino acids 642 to 677 (36 amino acids long) designated Spa64 (SEQ ID NO:45); amino acids 642 to 676 (35 amino acids long) designated Spa65 (SEQ ID NO:46); amino acids 642 to 675 (34 amino acids long) designated Spa66 (SEQ ID NO:47); amino acids 642 to 674 (33 amino acids long) designated Spa67 (SEQ ID NO:48); amino acids 643 to 678 (36 amino acids long) designated Spa68 (SEQ ID NO:49); amino acids 643 to 677 (35 amino acids long) designated Spa69 (SEQ ID NO:50); amino acids 643 to 676 (34 amino acids long) designated Spa70 (SEQ ID NO:51); amino acids 643 to 675 (33 amino acids long) designated Spa71 (SEQ ID NO:52); amino acids 643 to 674 (32 amino acids long) designated Spa72 (SEQ ID NO:53); amino acids 645 to 678 (34 amino acids long) designated Spa73 (SEQ ID NO:54); amino acids 645 to 677 (33 amino acids long) designated Spa74 (SEQ ID NO:55); amino acids 645 to 676 (32 amino acids long) designated Spa75 (SEQ ID NO:56); amino acids 645 to 675 (31 amino acids long) designated Spa76 (SEQ ID NO:57); amino acids 645 to 674 (30 amino acids long) designated Spa77 (SEQ ID NO:58); amino acids 646 to 678 (33 amino acids long) designated Spa78 (SEQ ID NO:59); amino acids 646 to 677 (32 amino acids long) designated Spa79 (SEQ ID NO:60); amino acids 646 to 676 (31 amino acids long) designated Spa80 (SEQ ID NO:61); amino acids 646 to 675 (30 amino acids long) designated Spa81 (SEQ ID NO:62); amino acids 646 to 674 (29 amino acids long) designated Spa82 (SEQ ID NO:63); amino acids 647 to 678 (32 amino acids long) designated Spa83 (SEQ ID NO:64); amino acids 647 to 677 (31 amino acids long) designated Spa84 (SEQ ID NO:65); amino acids 647 to 676 (30 amino acids long) designated Spa85 (SEQ ID NO:66); amino acids 647 to 675 (29 amino acids long) designated Spa86 (SEQ ID NO:67); amino acids 647 to 674 (28 amino acids long) designated Spa87 (SEQ ID NO:68); or the fragments/deletion mutants of any of SEQ ID NOs: 22-68.

According to an embodiment, the Spa peptide molecules of the present invention are 11 and 38 amino acid long polypeptides, contain at least 5 contiguous amino acids of Spa310 (amino acids 641-679) (SEQ ID NO:3), and are capable of inhibiting cdk2 kinase activity. The invention includes compounds having a cdk2 activity inhibiting polypeptide having the following amino acid sequence based upon the native amino acid sequence of pRb2/p130 (SEQ ID NO:21): amino acids 675 to 685 (11 amino acids long and designated Spa53) (SEQ ID NO:69); or fragments/deletion mutants thereof.

The invention also contemplates one or more conservative amino acid substitutions, wherein the polypeptide maintains cdk2 inhibitory activity.

According to an embodiment, the invention also provides deletion mutants of SEQ ID NO: 1-SEQ ID NO: 15 and SEQ ID NO:22-SEQ ID NO:69. In one embodiment, mutants of the present invention that are capable of exhibiting cdk2 inhibitory activity can be defined by generating deletion mutants beginning at the amino-terminus and/or COOH terminus of the full-length sequence of pRb2/p130 spacer domain. In addition, chimeric fusion proteins corresponding to these mutant sequences are also part of the invention.

For achieving cdk2 inhibitory activity and the associated growth suppressive effects, the Spa peptide molecules can be used as such or fused to a second polypeptide or conjugated to an agent. According to a preferred embodiment of the present invention, a given Spa peptide molecule is fused to a second polypeptide (e.g., the HIV Tat-derived peptide with the sequence GRKKRRQRRR (SEQ ID NO: 16), glutathione S-transferase, His-tag) via, for example, a disulfide bond, a thio-ether linkage or a peptide bond. In another embodiment, the peptide is conjugated to an agent. The agent may include, but is not limited to a toxin, a radioactive molecule, a detectable label, an imaging agent, a diagnostic agent, a chemotherapeutic agent, an immunomodulatory agent, and/or a translocating agent. The translocating agent can be used to translocate the peptide or preferably a therapeutic agent attached to the peptide into the cell in order to deliver the therapeutic agent to the cell. According to another embodiment, the peptide may be used together with an agent that functions in the cytoplasmic compartment of a cell, such as for example an agent that inhibits the cytoskeleton, or inhibits spindle formation. Several of these latter types of agents are known to be chemotherapeutic agents. In yet another embodiment, the peptide may be conjugated to another peptide such as one with binding specificity for cdk2. In another embodiment, the composition includes the peptide with a liposome or viral particle (e.g., for delivery in gene therapy).

The functional equivalents of Spa peptides can include peptidomimetics. In one embodiment, the functional equivalent may be chosen from a phage library member, a synthetic peptide library member, a combinatorial chemical library member, and a peptide mimetic.

According to an embodiment, the composition may further contain a pharmaceutically acceptable carrier, and optionally, the peptide or functional equivalent thereof is present in an effective amount. In other embodiments, the composition further contains another therapeutic agent including but not limited to an anti-cancer agent. The composition may be provided in a delivery vehicle well-known in the art, preferably in a sustained release form. In another aspect, a pharmaceutical preparation is provided having one or a combination of the afore-mentioned compositions and a pharmaceutically acceptable carrier. The pharmaceutical preparation and compositions may be in a sustained release vehicle.

According to an embodiment, the invention also provides for isolated nucleic acid molecules that code for Spa peptides and a composition containing the same. Thus, in yet another aspect, an isolated nucleic acid molecule is provided having (a) a nucleic acid molecule which codes for a peptide having an amino acid sequence of SEQ ID NOs: 1-15 and SEQ ID NOs:22-69, preferably, SEQ ID NO: 1, SEQ ID NO: 2, or SEQ ID NO: 3 or functionally equivalent fragments thereof, (b) degenerates of (a); and (c) full-length complements of (a) and (b). Using the amino acid sequence of SEQ ID NO: 1, SEQ ID NO: 2, or SEQ ID NO: 3 provided herein, one of ordinary skill in the art can readily determine the nucleic acid sequences that are degenerates thereof. According to an embodiment, the invention further provides an expression vector comprising the afore-mentioned isolated nucleic acid molecule, preferably operably linked to a promoter, and host cells and/or proliferating cells transformed or transfected with the expression vectors.

In another aspect, the invention provides a method for preventing or treating a disorder (e.g., neoplasms or tumor cell proliferation) characterized by cdk2 overexpression. The method can be used to prevent the disorder in a subject at risk of developing the disorder or, alternatively, to treat the disorder in a subject having the disorder. In embodiments of either, the methods further comprise first selecting a subject to be treated (e.g., a subject having the disorder or a subject at risk of developing the disorder). The method involves administering to a subject in need of such treatment a Spa peptide that inhibits cdk2 activity. In certain preferred embodiments, the Spa peptide may have an amino acid sequence of SEQ ID NO: 1, SEQ ID NO:2, and/or SEQ ID NO:3, or functional equivalents thereof.

According to an embodiment, the Spa peptide or functional equivalent thereof may be administered in an amount effective to inhibit the disorder. In other embodiments, the method may involve co-administering an anti-cancer agent to the subject. In these latter embodiments, the peptide and the anti-cancer agent are co-administered in a combined effective amount to inhibit the disorder. In related aspects of the foregoing methods non-peptide small molecules that functionally and/or structurally mimic the Spa peptides of the invention can also be used in place of the Spa peptides.

In one embodiment, the disorder is in or is likely to be in a tissue such as, but not limited to the lung, brain, breast, ovary, uterus, cervix, gastrointestinal tissue, colon, stomach, and bladder. In important embodiments, the disorder is a cancer. The cancer may be a primary tumor or a metastasis. The cancer may include but is not limited to lung cancer including non-small lung cancer, breast cancer, ovarian cancer (including endometrioid carcinoma), osteosarcoma, cervical cancer, colorectal cancer (e.g., colorectal adenomas and adenocarcinomas), thyroid cancer, prostate cancer, stomach cancer, and bladder cancer.

According to an embodiment, the peptide may be administered systemically. In another embodiment, the peptide may be administered locally. In yet another embodiment, the peptide may be administered in a plurality of administrations. In another embodiment, the method further involves administering to the subject an anti-cancer agent. The invention further provides a method for inhibiting/preventing tumor cell metastasis by administering to a subject in need of such treatment one or a combination of any of the afore-mentioned peptides or functional equivalents in an amount effective to prevent the formation or development of a metastasis. The metastasis may be present in, but is not limited to bone marrow, lung, brain, and/or liver.

According to an embodiment, the invention provides a method for detecting cells characterized by cdk2 overexpression involving delivering a Spa peptide, that in some embodiments has an amino acid sequence selected from the group consisting of SEQ ID NO:1, SEQ ID NO:2, and SEQ ID NO:3 or functional equivalent thereof, to cells, and evaluating cell cycle distribution to determine the distribution of cells through the G1, S and or G2/M phases of the cell cycle, wherein cells exposed to the Spa peptide being present in G1 phase of the cell cycle in greater percentage than control cells (i.e., cells not exposed to the Spa peptide) is indicative of cdk2 overexpression by the cells.

In one embodiment, the delivering occurs in vivo and the peptide is administered to a subject either systemically or locally. In some embodiments, the peptide is conjugated to a detectable label. The detectable label may include, but is not limited to a radioisotope, a contrast agent, and/or a gaseous agent.

In one embodiment, the cells are breast tissue cells. In another embodiment, the cells are present in a population selected from the group consisting of bone marrow tissue, lung tissue, brain tissue, and liver tissue. In a related embodiment, the cells are harvested from a subject having a disorder characterized by cdk2 over-expression, prior to treating the subject with gene therapy or radiation or chemotherapy. The disorder characterized by cdk2 over-expression may be non-small cell lung cancer, but is not so limited.

According to an embodiment, the present invention includes a method for identifying a compound that interferes with or inhibits interaction between cdk2 and a Spa peptide (that preferably comprises an amino acid having a sequence of SEQ ID NO: 1, SEQ ID NO:2, or SEQ ID NO:3, or functional equivalent thereof). The method may involve the following steps: performing a first assay between cdk2 and the peptide or functional equivalent thereof to obtain a first assay result; performing a second assay between cdk2 and the peptide or functional equivalent thereof in the presence of a compound to obtain a second assay result; and comparing the first and second assay results to determine whether the compound inhibits interaction between cdk2 and the peptide or functional equivalent thereof. The method may also include a negative pre-screen in which compounds are initially tested and negatively selected based on their ability to bind to cyclins and/or cdks. In one embodiment, the compound is a molecular library member. The molecular library may include, but is not limited to a peptide library such as a phage display peptide library, a peptidomimetic library, a combinatorial chemistry library, a synthetic peptide library, and a natural compound library.

The compounds of the present invention may be administered to persons having cancer conditions as treatment by any of the administration methods that are known in the art. These include: systemic or local (intramuscular, intravenous or tumoral injection); oral (liquid, pill, capsule); inhalation (see e.g., U.S. Pat. Nos. 6,682,716; 6,610,653; 6,503,481; and 5,952,008); buccal (see e.g., U.S. Pat. Nos. 5,284,657 and 5,624,677); eye drops (see e.g., U.S. Pat. No. 5,283,236); topical (e.g. cream, gel, ointment) (see e.g., U.S. Pat. No. 6,595,947); suppository; or transdermal (e.g. patch) (see e.g., U.S. Pat. Nos. 7,315,758 and RE39,588). It may be desirable to adapt these well-known techniques of administration to suit the particular type of cancerous tissue, its location, or other circumstances. This can be done using well-established techniques and principles in the art.

Similarly, compounds of the present invention may be coupled with any transport molecule and/or delivery system known in the art. See U.S. Pat. Nos. 7,067,487 and 6,916,489. Any technique known in the art for transport of an amino acid of the present invention into a cell is also envisioned; e.g. microbubbles (see e.g., Tsutsui, et al., 2004, Cardiovascular Ultrasound, 2, 23); polymer-based micelles (micelles made of amphiphilic block co-polymers) (see e.g., Rangel-Yagui, et al., 2005, J Pharm Pharmaceut Sci (www.cspscanada.org) 8(2):147-163 (and references 78-81 cited therein); fatty acid/synthetic surfactant mixed micelles (see e.g., Vladimir P. Torchilin, Journal of Controlled Release 73 (2001) 137-172 (and references cited therein); liposomes and liposome-like aggregates (see e.g., Weissig, et al., Pharmaceutical Research, 15 (1998), 1552-1556 and Weissig, et al., Pharmaceutical Research, 15 (1998), 334-337); and microcapsules and microspheres. The use of these transport molecules and systems may be adapted to suit the particular type of cancerous tissue, its location, or other circumstances by using well-established techniques and principles in the art.

### EXAMPLES

The following working examples are provided to demonstrate preferred embodiments of the invention, but of course, should not be construed as in any way limiting the scope of the present invention. The examples below were carried out using conventional techniques that are well-known and routine to those of skill in the art, except where otherwise described in detail. Further, it should be appreciated by those of skill in the art that the techniques disclosed in the examples represent techniques found by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Deletion mutants of pRb2/p130 Spacer Region and their Cdk2 Inhibitory Activity

Different portions of the pRb2/p130 spacer region were prepared, expressed as GST-fusion proteins and assayed for their kinase inhibitory activity as follows:

Cell culture and transfections: The NIH/3T3 cell line was obtained from the American Type Culture Collection (ATCC, Rochville, Md.) and was grown at 37° C., in a 5% CO2/95% atmosphere, in Dulbecco's modified Eagle's medium (Mediatech Inc., Herndon, Va.) supplemented with fetal bovine serum (FBS) (Mediatech Inc., Herndon, Va.). Transfections were performed using the Fugene transfectant reagent (Roche Applied Science, Indianapolis, Ind.) according to the manufacturer's protocol.

Constructs preparation: A prokaryotic expression vector pGEX-2T (Stratagene Inc., La Jolla Calif.) and polymerase chain reaction (PCR) were used to generate chimeric Glutathione-S-Transferase constructs. The primers used by PCR to amplify the fragments that were subcloned in the pGEX-2T were derived from the 5′ and 3′ ends of different regions of the spacer domain of pRb2 μl 30. The nucleotide and amino acid positions, and the fragment length in base pairs and amino acids are shown in Table 1. The mammalian expression vector pEF6/V5-His TOPO (Invitrogen Corp, Carlsbad, Calif.) was used to generate the constructs pEF6/V5-His-spacer and pEF6/V5-His-310 that express the correspondent genes with a C-terminal V5 epitope. The spacer and the 310 fragments were PCR-amplified with the respective primers (Table 1) to generate 3′ends of the cDNAs without stop codons and were subsequently subcloned into pEF6/V5-His TOPO (Invitrogen Corp, Carlsbad, Calif.) according to the manufacturer's instructions to obtain cDNAs coding for C-terminal fusion proteins. All generated constructs were confirmed by automated sequencing. Information regarding the primers is available upon request.

GST Fusion protein preparation: XL1-Blue bacteria carrying the pGEX-2T vectors were grown to mid log phase and then induced to express protein by adding 0.25 mM of isopropyl-1-thio-β-D-galactopyranoside (IPTG, Roche Applied Science, Indianapolis, Ind.). The cultures were shaken for 4 h; bacteria were then pelleted and resuspended in NENT buffer (20 mM Tris pH 8, 100 mM NaCl, 1 mM EDTA, 0.5% NP-40). Cell suspensions were sonicated, pelleted and the supernatant collected. The remaining bacteria were then resuspended in NENT buffer plus 2% of N-Lauryl-Sarcosine, pelleted and the supernatants were collected again. The combined supernatants were incubated with Glutathione agarose (Amersham Biosciences, Piscataway, N.J.) overnight at 4° C. The agarose was then pelleted and washed three times in NENT buffer.

Kinase assays: Cell lysates from NIH/3T3 were prepared by resuspending pelleted cells in 500 μl of lysis buffer (50 mM Tris, 5 mM EDTA, 250 mM NaCl, 50 mM NaF, 0.1% Triton, 0.1 mM Na₃VO₄, plus protease inhibitors). An equal amount of protein for each fraction (100 μg) was immunoprecipitated with a polyclonal anti-cdk2 antibody (De Luca et al., 1997, J Biol Chem, 272, 20971-4). The complexes were pulled down with protein A-Sepharose and washed three times with lysis buffer and twice with lysis buffer containing 400 mM NaCl. The complexes were equilibrated in kinase assay buffer (20 mM HEPES pH 7.4, 10 mM MgAc, 20, 1 mM DTT). In order to detect the in vitro inhibition of cdk2 activity, each sample was incubated with an equal amount (0.1 μg) of each pGEX-2T fusion protein, in a final volume of 20 μl of kinase buffer, using 5 μCi/sample of γ-ATP (Amersham Biosciences, Piscataway, N.J.) and 2 μg of Histone H1 for 30 minutes at 30° C. Kinase assays were repeated at least three times, giving an inter assay standard deviation within 10% after normalization for protein amount. In order to evaluate the in vivo inhibition of cdk2 activity, cdk2 was immunoprecipitated, as described above, from NIH/3T3 cells transiently transfected with 5 μg of pEF6/V5-spacer, pEF6/V5-Spa310 or the vector alone as a control. The immunocomplexes were split in two tubes. A half of each sample was assayed for cdk2 activity in a final volume of 20 μl of kinase buffer with 5 μCi/sample of γ-ATP and 2 μg of Histone H1 for 30 min. at 30° C.; the second one was tested in western blot analysis to confirm that all the samples contained the amount of cdk2 immunoprecipitated. Briefly the samples were separated into 12% SDS polyacrylamide gels (SDS-PAGE), and then transferred into a nitrocellulose membrane (Schleicher & Schuell, Germany). The membrane was blocked with 5% non-fat dry milk in 1×TBST and incubated with the polyclonal anti-cdk2 antibody described above. Anti-rabbit peroxidase conjugated (1:10,000) (Amersham, Ill.) and ECL detection system (Enhanced Chemiluminescence Kit; Du Pont NEN, Boston, Mass.) were used for the detection.

Cdk2 inhibitory activity of the fragments of pRb2/p130 spacer domain: Presented in Table 1 are several constructs containing different portions of the pRb2/p130 spacer region that were prepared, expressed as GST-fusion proteins and assayed for their kinase inhibitory activity. Cdk2 immunocomplexes were precipitated from lysates of exponentially growing NIH/3T3 cells. Each GST fusion protein was added to the immunoprecipitate and the mixture was subjected to a kinase assay using histone H1 as a substrate. Three GST-fusion proteins containing different regions of the spacer domain (Spa, Spb and Spc) were created. We found that an inhibitory effect on histone H1 phosphorylation was exerted only by the first segment (Spa) (see Table 1). Three additional mutants derived from the Spa construct (Spa1, Spa2 and Spa3) were developed and tested. We found that the constructs Spa1, Spa2 and Spa3 proved to have little or no inhibitory effect on the cdk2 activity compared to the immunoprecipitates treated with GST alone and the GST-spacer fusion protein. Therefore, two other constructs (Spa12 and Spa23) overlapping the central part of the Spa region were generated and tested. Since only the GST-fusion protein containing the Spa23 fragment was able to inhibit cdk2 activity, several constructs were finally developed and tested for kinase inhibitory activity by deleting segments of the 5′ and 3′ ends of the Spa23 (Spa72, Spa92, Spa38, Spa311, Spa310 and Spa312).

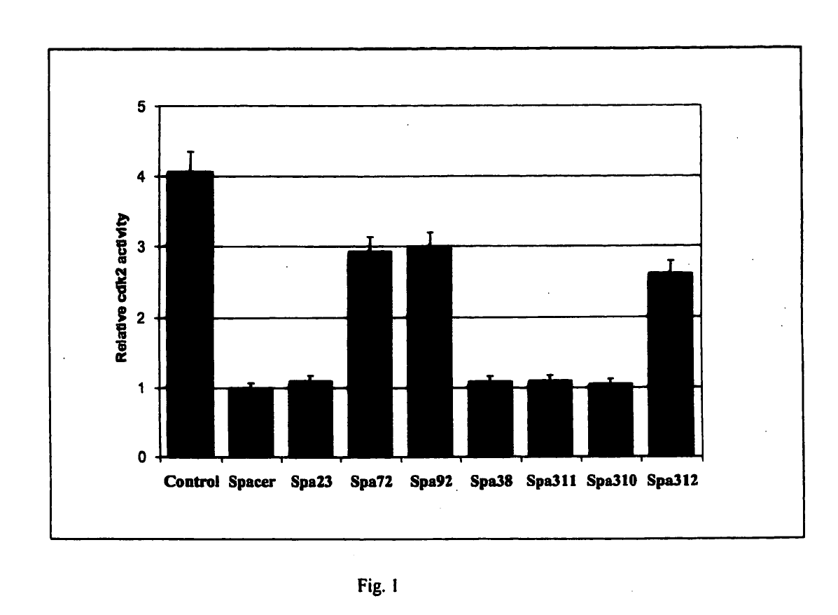

FIG. 1 shows the effects on cdk2 activity exhibited by the seven final constructs. The Spa310 construct, spanning the region between the amino acids 641 and 679 (39 amino acids), represented the smallest molecule able to maintain the specific inhibitory ability of the pRb2/p130 spacer domain on cdk2 activity in vitro. The relative kinase activity values shown represent an average of three independent experiments.

TABLE 1

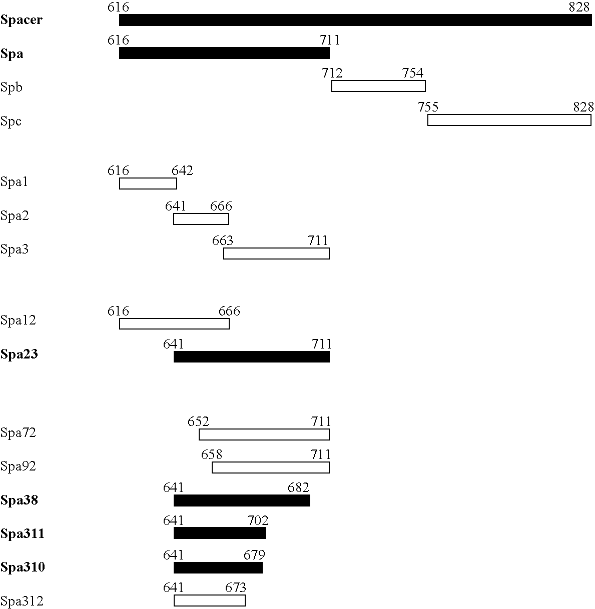

| Construct | Primer | | Nucleotides | Base Pair | Amino Acids | Amino Acids | kDa | Kinase |
|---|---|---|---|---|---|---|---|---|
| pGEX-2T | 5′ | 3′ | from to | in length | from to | in length | SDS gel | assay |
| Spacer | Sp1 | Sp2 | 1915-2550 | 636 | 616-828 | 212 | 25.4 | P |
| Spa | Sa1 | Sa2 | 1915-2202 | 288 | 616-711 | 96 | 11.5 | P |
| Spb | Sb1 | Sb2 | 2203-2331 | 129 | 712-754 | 43 | 5.2 | N |
| Spc | Sc1 | Sc2 | 2332-2550 | 219 | 755-828 | 73 | 8.8 | N |
| Spa1 | Sa1 | Sa6 | 1915-1995 | 81 | 616-642 | 27 | 3.2 | N |
| Spa2 | Sa3 | Sa4 | 1990-2067 | 78 | 641-666 | 26 | 3.1 | N |
| Spa3 | Sa5 | Sa2 | 2056-2202 | 147 | 663-711 | 49 | 5.8 | N |
| Spa12 | Sa1 | Sa4 | 1915-2067 | 153 | 616-666 | 51 | 6.1 | N |
| Spa23 | Sa3 | Sa2 | 1990-2202 | 213 | 641-711 | 71 | 8.5 | P |
| Spa72 | Sa7 | Sa2 | 2093-2202 | 180 | 652-711 | 60 | 7.3 | N |
| Spa38 | Sa3 | Sa8 | 1990-2175 | 186 | 641-702 | 62 | 7.4 | P |
| Spa311 | Sa3 | Sa11 | 1990-2115 | 126 | 641-682 | 42 | 5.0 | P |
| Spa310 | Sa3 | Sa10 | 1990-2106 | 117 | 641-679 | 39 | 4.7 | P |
| Spa312 | Sa3 | Sa12 | 1990-2088 | 99 | 641-673 | 33 | 3.9 | N |

Nucleotide and amino acid positions, and the fragment length (in base pairs and amino acids) of deletion mutants based on the sequence of the pRb2/p130 spacer domain are shown. The constructs listed in bold, also represented with the black bars, demonstrated a positive inhibitory effect on cdk2 activity. No significant inhibitory effect on cdk2 activity was detected in the other constructs. P=positive, N=negative.

### Example 2

### Localization of Spacer and Spa310 Molecules in Cells

Expression and localization of Spacer and Spa310 molecules in cells were evaluated as follows:

Immunofluorescence: Exponentially growing NIH/3T3 cells were seeded on two-well micro-chamber slides (Nunc, Naperville, Ill.) and transfected the next day with 1 μg of pEF6/V5-spacer, pEF6/V5-Spa310 or the vector alone (pEF6/V5) as a control. Forty-eight hours after transfection, cells were fixed in PBS-buffered 4% paraformaldehyde for 15 min at room temperature and then permeabilized in 0.1% Triton-X 100/PBS before washing and blocking in 0.1% BSA/0.1% Triton-X 100/PBS in 370 C for 30 min. The primary antibody used for immunofluorescence was an anti-V5 monoclonal antibody (Invitrogen Corp, Carlsbad, Calif.) that recognizes the epitope present in the constructs. The secondary antibody was a goat anti-mouse conjugated with Alexa-568 (Molecular Probes, Eugene, Oreg.) used at a dilution of 1:2000. DNA was counterstained with DNA fluorochrome 4′,6′ diamedino-2-phenylindole (DAPI, Sigma Inc., St. Louis, Mo., USA) and slides were mounted with the SlowFade anti-fade reagent (Molecular Probes, Eugene, Oreg.). Negative controls were performed with secondary antibodies only. The slides were seen under an inverted Olympus IX70 microscope (Olympus America, Inc. Melville, N.Y.). Fluorescence images were captured with Sensicam QE camera (Cooke Co., Auburn Hills, Mich.) and operated with SlideBook 3.0 software (Intelligent Imaging Innovations Inc., Denver, Colo.) in order to eliminate the background haze and reveal individual foci.

The cells, 48 h after their transfection, were analyzed for immunofluorescence localization of spacer and Spa310 expression, using a monoclonal antibody that specifically recognizes the V5 tag carried by the transfected constructs. Immunofluorescence analysis of transfected cells with anti-V5 antibody revealed a localization of the tagged Spacer and Spa310 proteins in the cytoplasm and in the nucleus, observed as red spots (photograph not presented). Cells were counterstained with DNA fluorochrome 4′, 6′ diamedino-2-phenylindole (DAPI, Simga Inc., St. Louis, Mo., USA) to visualize the nuclei seen as blue spots.

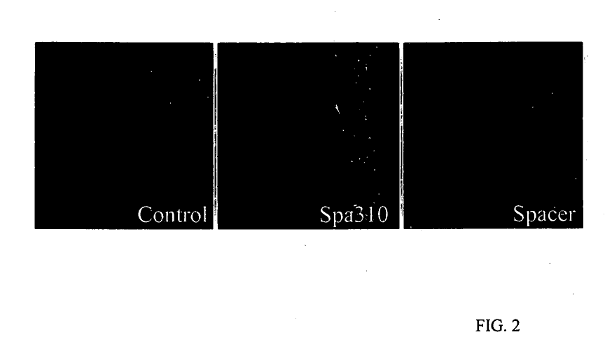

FIG. 2 shows that the tagged proteins are strongly expressed in the transfected NIH/3T3 cells. The molecules' expression was detected both in the cytoplasm and in the nucleus (red foci) of the transfected cells. The red foci were absent in cells transfected with the control vector, indicating specificity of immunostaining. Similarly, red foci were absent in non-transfected cells. The data shown are representative of three independent experiments.

### Example 3

### Effect of Spa310 on Endogenous Cdk2 Activity

From the above, it can be seen that pRb2/p130 spacer and Spa310 strongly and similarly inhibited cdk2 activity in vitro. In this example, the effect pRb2/p130 spacer and Spa310 on endogenous cdk2 activity is described. The NIH/3T3 cells were transiently transfected with pEF6/V5-spacer, pEF6/V5-Spa310 or the control vector pEF6/V5. At 48 h after transfection, endogenous cdk2 was immunoprecipitated and assayed for kinase activity using histone H1 as a substrate.

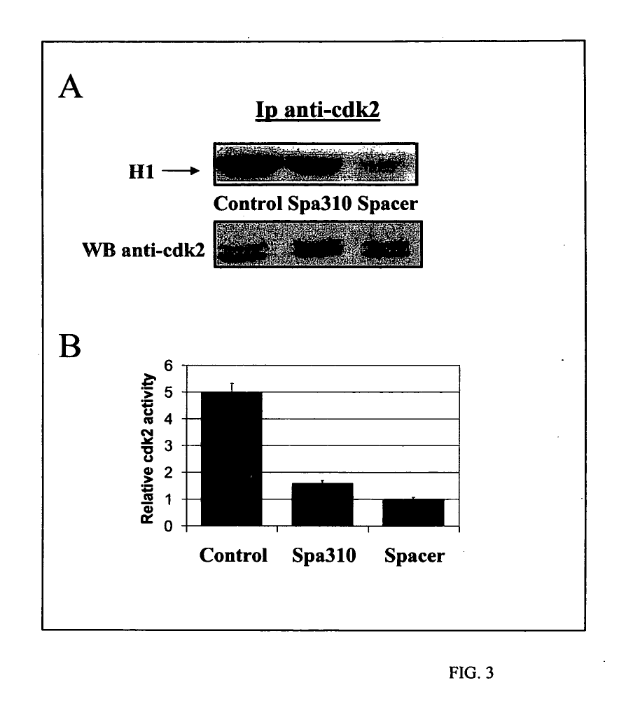

FIGS. 3a and 3b show a significant inhibition of cdk2-dependent histone phosphorylation mediated by both the spacer domain and the Spa310 small peptide expression. In FIG. 3a, asynchronously growing NIH/3T3 cells were transfected with pEF6/V5 vector, pEF6/V5-Spa310 or pEF6/V5-spacer. Forty-eight hours after transfection, cdk2 was immunoprecipitated with anti-cdk2 polyclonal antibody. The immunocomplexes were extensively washed, and split into two tubes. The first half was incubated in kinase assay reaction buffer with 2 μg of histone H1 as a substrate and resolved on 10% gel. Phosphorylation of histone H1 was monitored by autoradiography. The second half was used in western blot analysis to test that the amount of cdk2 immunoprecipitated is the same for all the samples. In FIG. 3b, the relative kinase activity values shown represent an average of three independent experiments.

### Example 4

### Demonstration of Suppression of Cell Growth by Spa310 Observed by Colony Formation Assay

Residues critical for the inhibition of cdk2 activity were identified as described above. The crucial role of cdk2 activity is known to induce the expression of cell cycle-regulatory genes, thus leading to cell cycle progression (Chae et al., 2004, Oncogene, 23, 4084-8; Yu et al., 2004, Biochem Pharmacol, 67, 1907-16). Therefore, inhibiting the phosphorylation ability of this kinase with the Spa310 small molecule should induce cell cycle arrest. In this example, it is shown that these residues are in fact sufficient for cell growth inhibition.

Colony formation assay: The effects of the pRb2/p130 spacer domain and 39 aa small peptide overexpression on cellular growth was examined by colony formation assays. Exponentially growing NIH/3T3 cells were seeded in 100-mm dishes at a density of 5×105 cells per 100 mm dish the day before transfection. Cells were transfected with 5 μg of pEF6/V5-spacer, pEF6/V5-Spa310 or the control vector pEF6/V5 carrying the brasticidin S-resistant gene (bsr) as a selectable marker gene. For selection, blasticidin S hydrochloride (Funakoshi, Tokyo, Japan) was added to the culture medium 48 h after transfection, at a final concentration of 10 μg/mL. After 6 days, 1×103 of blasticidin S-resistant cells in each well were plated in triplicate in 60-mm dishes, in order to evaluate their colony-forming ability. Cells were then incubated at 37° C. for eight days in a selecting medium containing blasticidin S hydrochloride at a final concentration of 10 μg/mL. Colonies, defined as groups of a minimum of 50 cells, were counted after staining with 2% methylene blue in 95% ethanol.

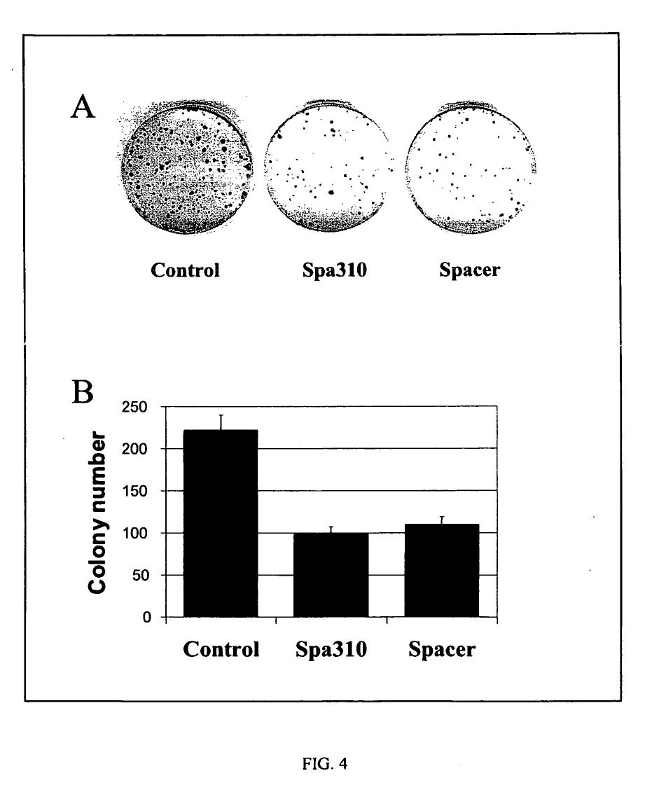

As presented in FIGS. 4a and 4b, the spacer region caused a decrease on the ability of the cells to form colonies of about 50% and the Spa310 of about 60%. The control vector alone had no effect on the colonogenic capacity of the cells, demonstrating that the small molecules are, rather than the entire spacer region, indeed sufficient for achieving the growth-inhibited phenotype. In FIG. 4a, NIH/3T3 cells were transfected with pEF6/V5 vector, pEF6/V5-Spa310 or pEF6/V5-spacer. Forty-eight hours after transfection, cells were selected with 10 μg/mL of blasticidin S hydrochloride. After six days, an equal number of resistant cells were plated in 60-mm dishes and colony-forming ability was evaluated after eight days of continuous selection. In FIG. 4b, the colony number values shown represent an average of three independent experiments.

### Example 5

### Effect of Spa310 on Cell Cycle Arrest

To demonstrate that Spa310 small molecule maintains the growth arrest properties, like the full-length Spacer, leading to an arrest in the G0/G1 phase of the cell cycle, cells were transiently transfected with 5 μg of pEF6/V5-spacer, pEF6/V5-Spa310 or the vector alone as a control. A marker plasmid expressing enhanced green fluorescent protein (EGFP)-spectrin was included in the transfection mixture. Control cells, which were not transfected with EGFP-spectrin, were used as the EGFP-negative population. Cells were harvested and examined for their cell cycle states by fluorescence-activated cell sorter (FACS) analysis. After harvest, cells were fixed by adding ice-cold 70% ethanol while vortexing. Fixed cells were stored at 4° C. for at least 30 min and then washed once with PBS. Cells were then stained with 10 μg/ml propidium iodide (Roche Applied Science, Indianapolis, Ind.), 250 μg/ml RNase (Sigma, St. Louis, Mo.) in PBS and incubated at 37° C. for 30 min in the dark. Transfected cells were gated according to their EGFP expression and the DNA content was determined by flow cytometry analysis. The percentage of cells in the different phases of the cell cycle was measured with a FACS Calibur instrument (Becton-Dickinson, San Jose, Calif.) and the data obtained were analyzed by WinMDI 2.8 software.

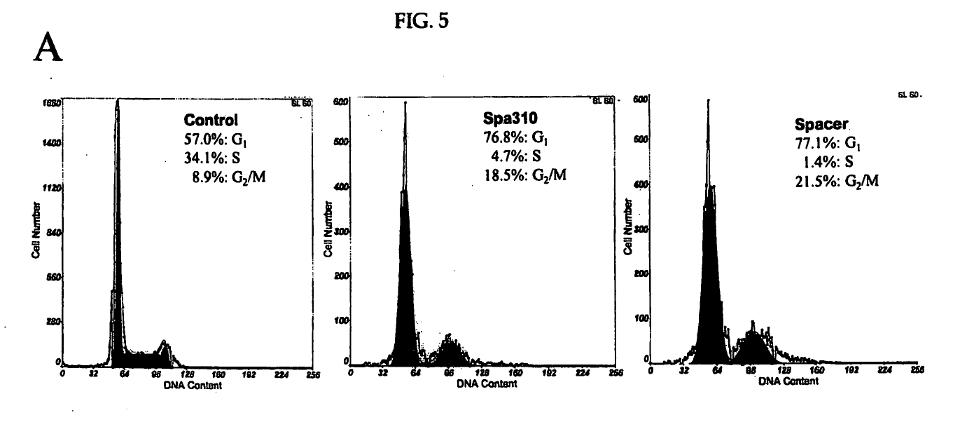

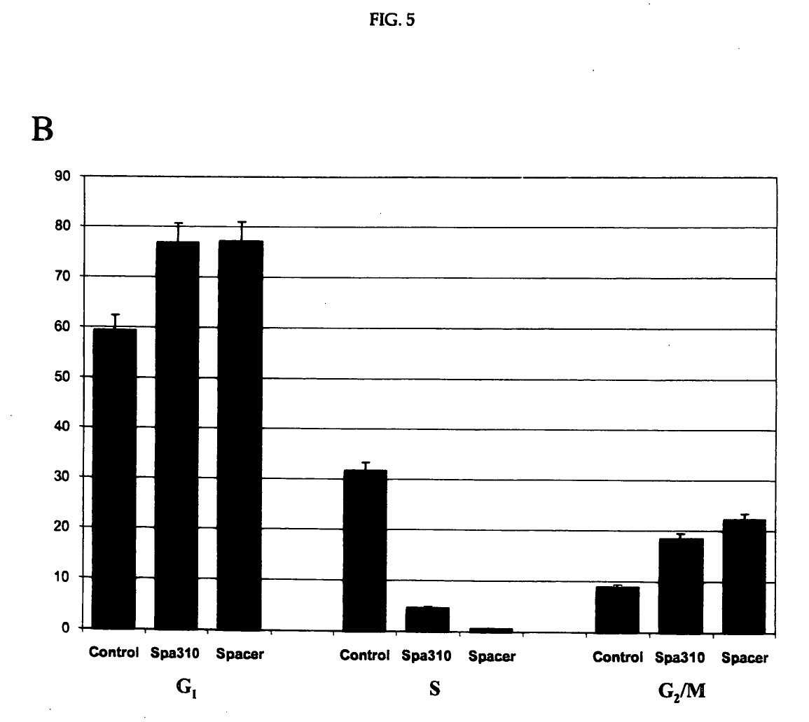

The inhibition of cdk2 activity caused by the spacer and the Spa310 small molecule had an effect on cell cycle distribution, ultimately leading to cell cycle arrest. Cell cycle analysis/flow cytometry analysis was performed in NIH/3T3 cells transiently transfected with pEF6/V5-spacer, pEF6/V5-Spa310 or the control vector pEF6/V5. A marker plasmid expressing enhanced green fluorescent protein, (EGFP)-spectrin was included in the transfection mixture. At 48 h after transfection, cells were fixed and stained with propidium iodide to determine the DNA content and were simultaneously examined for EGFP expression. Flow cytometry analyses indicated that the spacer and the Spa310 small molecule similarly induced a G0/G1 arrest of the cell cycle, reducing the population of cells in the S phase (FIGS. 5a and 5b).

In FIG. 5a, three plasmids were co-expressed with EGFP-spectrin, in a 1:10 ratio, in transient transfection assays. Cells were harvested 48 hours after transfection, stained with propidium iodide and analyzed by flow cytometric gating for both GFP and propidium iodide. Cell cycle distribution was evaluated to determine the distribution of cells through the G₁, S and G₂/M phases of the cell cycle. In FIG. 5b, the percentages shown represent an average of three independent experiments. Thus, it is shown that the Spa310 small molecule, similarly to the full-length spacer domain of pRb2/p130, acts as a growth suppressor by inducing a G₀/G₁ arrest of the cell cycle.

### Example 6

### Treatment of Tumor Growth In Vivo

In this example, it is shown that the Spa310 small molecule does suppress the tumor growth.

Cell Culture The A549 (wt p53, and wt RB) non-small cell lung cancer cell line was cultured in Ham's F-12 with NaHCO3 (0.75 g NaHCO3/500 ml of Ham's F-12) and 10% (vol/vol) fetal bovine serum (FBS). The cells were cultured at 37° C. in a humidified incubator containing 5% CO2. Cells were harvested, when they reached 70-80% confluence.

Preparation of Tumor Xenografts in Immunodeficient Mice: The animal experiments were carried out in accordance with the Guidelines for the Care and Use of Laboratory Animals (National Institute of Health publication number 85-23) and the institutional guidelines of Temple University. 4-6 week-old female athymic nude mice (CD1, nu/nu) were bought from Charles River Laboratory (Wilmington, Mass.). About a week after the mice arrived, the A549 cells were harvested and then suspended in unsupplemented culture medium without FBS. After the mice were anesthetized by isoflurane inhalation, 4×106 cells in a final volume of 100 μl were injected subcutaneously into the right flank of each mouse by using 1-cc syringes with 25.5-gauge needles.

In Vivo Treatments: The length and width of each tumor were measured every time in triplicates by using a Vernier caliper. Then the median of the measurements was applied to the following formula to calculate the tumor volume: Tumor volume=(the length of the tumor×the width of the tumor)2/2. When the tumors grew up to a volume of about 0.05 cm³, the mice were treated with the peptides. 4 peptides were tested (Spa310, Spa310-Tat, Scramble, and Scramble-Tat). The amino acid sequence of each of the peptides is as follows:

TAT-Spa310:

(SEQ ID NO:17)

G R K K R R Q R R R P P L T P R R V T E V R A D T
G G L G R S I T S P T T L Y D R Y S S P P A S T T
R (51 amino acids);
TAT-SCRAMBLE:

(SEQ ID NO:18)

G R K K R R Q R R R P P T D Y S P A T R S V G I T
R P T L P T S R D S Y T G R E R S V G P L R T A L
T (51 amino acids);
Spa310:

(SEQ ID NO:3)

L T P R R V T E V R A D T G G L G R S I T S P T T
L Y D R Y S S P P A S T T R (39 amino acids);
and
SCRAMBLE:

(SEQ ID NO:19)

T D Y S P A T R S V G I T R P T L P T S R D S Y T
G R E R S V G P L R T A L T (39 amino acids).

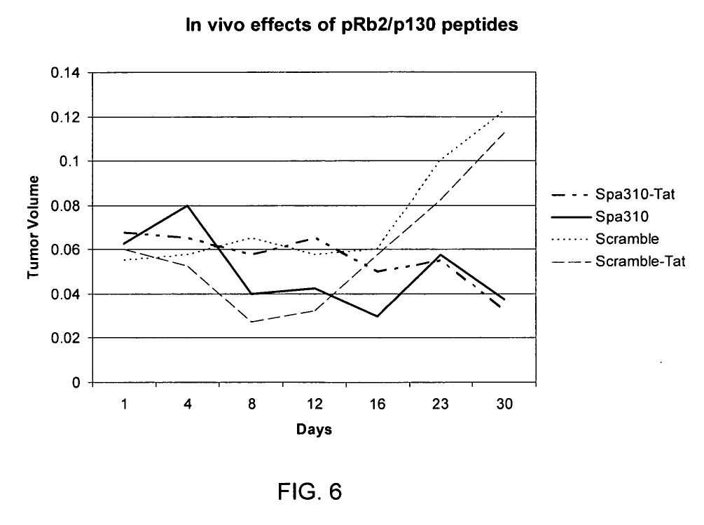

For each peptide type, three mice were treated. 50 μl of each peptide type at 1.5 mM in distilled water were injected into the tumoral region every 4 days in the first 5 treatments, and then every 7 days in the last 2 treatments. The tumor sizes were measured before each treatment. Tumor volumes were calculated and plotted on a graph. Tumor suppressive effects of four different peptides (TAT-Spa310, TAT-SCRAMBLE, Spa310 and SCRAMBLE) injected into the mice were as illustrated in FIG. 6.

Thus, it has been demonstrated herein that the small peptide molecules of the present invention are able to inhibit cdk2 activity, inhibit cell cycle progression and induce growth arrest when expressed in cells and promote tumor regression in vivo. The invention, however, should not be limited to the peptides exemplified above. In fact, it may prove to be the case that the most useful pharmacological small molecule peptides and nucleic acids designed and synthesized in light of this disclosure will be second generation derivatives of the exemplified molecules.

All publications, patents and patent applications mentioned in the specification are indicative of the level of those skilled in the art to which this invention pertains. All publications, patents and patent applications are herein incorporated by reference (including the references cited therein) to the same extent as if each individual publication or patent application was specifically and individually indicated to be incorporated by reference. Although the foregoing invention has been described in some detail by way of illustration and example for purposes of clarity of understanding, it will be obvious that certain changes and modifications may be practiced within the scope of the appended claims.

1-11. (canceled)

12. A polypeptide comprising a fragment of the full-length pRB2/p130 spacer domain (616-828) or a variant of the fragment, wherein the fragment or the variant is between 9 and 38 amino acids long, wherein the fragment has at least 9 contiguous amino acids of Spa310 (641-679), and wherein the polypeptide is capable of inhibiting cdk2 kinase activity.

13. A polypeptide consisting essentially of a fragment of the full-length pRB2/p130 spacer domain (616-828) or a variant of the fragment, wherein the fragment or the variant is between 9 and 38 amino acids long, wherein the fragment has at least 9 contiguous amino acids of Spa310 (641-679), and wherein the polypeptide is capable of inhibiting cdk2 kinase activity.

14. The polypeptide of claim 12 or 13, wherein the fragment is selected from the group consisting of SEQ ID NO:22 (9 amino acids long and designated Spa40); SEQ ID NO:23 (11 amino acids long and designated Spa41); SEQ ID NO:24 (14 amino acids long and designated Spa42); SEQ ID NO:25 (25 amino acids long and designated Spa43); SEQ ID NO:26 (30 amino acids long and designated Spa44); SEQ ID NO:27 (36 amino acids long and designated Spa45); SEQ ID NO:28 (14 amino acids long and designated Spa46); SEQ ID NO:29 (19 amino acids long and designated Spa47); SEQ ID NO:30 (24 amino acids long and designated Spa48); SEQ ID NO: 31 (29 amino acids long and designated Spa49; SEQ ID NO:32 (34 amino acids long and designated Spa50); SEQ ID NO:33 (20 amino acids long and designated Spa51); SEQ ID NO:34 (9 amino acids long and designated Spa52); SEQ ID NO:39 (38 amino acids long and designated Spa58); SEQ ID NO:40 (37 amino acids long and designated Spa59); SEQ ID NO:41 (35 amino acids long and designated Spa60); SEQ ID NO:42 (34 amino acids long and designated Spa61); SEQ ID NO:43 (33 amino acids long and designated Spa62); SEQ ID NO:44 (37 amino acids long and designated Spa63); SEQ ID NO:45 (36 amino acids long and designated Spa64); SEQ ID NO:46 (35 amino acids long and designated Spa65); SEQ ID NO:47 (34 amino acids long and designated Spa66); SEQ ID NO:48 (33 amino acids long and designated Spa67); SEQ ID NO:49 (36 amino acids long and designated Spa68); SEQ ID NO:50 (35 amino acids long and designated Spa69); SEQ ID NO:51 (34 amino acids long and designated Spa70); SEQ ID NO:52 (33 amino acids long and designated Spa71); SEQ ID NO:53 (32 amino acids long and designated Spa72); SEQ ID NO:54 (34 amino acids long and designated Spa73); SEQ ID NO:55 (33 amino acids long and designated Spa74); SEQ ID NO:56 (32 amino acids long and designated Spa75); SEQ ID NO:57 (31 amino acids long and designated Spa76); SEQ ID NO: 58 (30 amino acids long) designated Spa77); SEQ ID NO:59 (33 amino acids long and designated Spa78); SEQ ID NO:60 (32 amino acids long and designated Spa79); SEQ ID NO:61 (31 amino acids long and designated Spa80); SEQ ID NO: 62 (30 amino acids long and designated Spa81); SEQ ID NO:63 (29 amino acids long and designated Spa82); SEQ ID No:64 (32 amino acids long and designated Spa83); SEQ ID NO:65 (31 amino acids long and designated Spa84); SEQ ID NO:66 (30 amino acids long) designated Spa85); SEQ ID NO: 67 (29 amino acids long and designated Spa86); and SEQ ID NO:68 (28 amino acids long and designated Spa87).

15. A polypeptide comprising a fragment of the full-length pRB2/p130 spacer domain (616-828) or a variant of the fragment, wherein the fragment or the variant is capable of inhibiting cdk2 kinase activity and wherein the fragment is selected from the group consisting of SEQ ID NO:35 (39 amino acids long and designated Spa54), SEQ ID NO: 36 (39 amino acids long and designated Spa55); SEQ ID NO:37 (39 amino acids long and designated Spa56); and SEQ ID NO:38 (39 amino acids long and designated Spa57).

16. The polypeptide of claim 12 or 13, wherein the polypeptide is a fusion polypeptide.

17. The polypeptide of claim 12 or 13, wherein the polypeptide is conjugated to an agent.

18. A polypeptide consisting essentially of a fragment of the full-length pRB2/p130 spacer domain (616-828) or a variant of the fragment, wherein the fragment or the variant is capable of inhibiting cdk2 kinase activity, and wherein the fragment is selected from the group consisting of SEQ ID NO:35 (39 amino acids long and designated Spa54), SEQ ID NO:36 (39 amino acids long and designated Spa55); SEQ ID NO:37 (39 amino acids long and designated Spa56); and SEQ ID NO:38 (39 amino acids long and designated Spa57).

19. A fragment of the full-length spacer domain amino acid sequence of pRB2/p130 (616-828) or a variant thereof, wherein the fragment or the variant is between 9 and 38 amino acids in length, wherein the fragment has at least 9 contiguous amino acids of Spa310 (641-679), and wherein the fragment is capable of inhibiting cdk2 kinase activity, wherein.

20. The fragment of claim 19, wherein the fragment is selected from the group consisting of SEQ ID NO:22 (9 amino acids long and designated Spa40); SEQ ID NO:23 (11 amino acids long and designated Spa41); SEQ ID NO:24 (14 amino acids long and designated Spa42); SEQ ID NO:25 (25 amino acids long and designated Spa43); SEQ ID NO:26 (30 amino acids long and designated Spa44); SEQ ID NO:27 (36 amino acids long and designated Spa45); SEQ ID NO:28 (14 amino acids long and designated Spa46); SEQ ID NO:29 (19 amino acids long and designated Spa47); SEQ ID NO:30 (24 amino acids long and designated Spa48); SEQ ID NO: 31 (29 amino acids long and designated Spa49; SEQ ID NO:32 (34 amino acids long and designated Spa50); SEQ ID NO:33 (20 amino acids long and designated Spa51); SEQ ID NO:34 (9 amino acids long and designated Spa52); SEQ ID NO:39 (38 amino acids long and designated Spa58); SEQ ID NO:40 (37 amino acids long and designated Spa59); SEQ ID NO:41 (35 amino acids long and designated Spa60); SEQ ID NO:42 (34 amino acids long and designated Spa61); SEQ ID NO:43 (33 amino acids long and designated Spa62); SEQ ID NO:44 (37 amino acids long and designated Spa63); SEQ ID NO:45 (36 amino acids long and designated Spa64); SEQ ID NO:46 (35 amino acids long and designated Spa65); SEQ ID NO:47 (34 amino acids long and designated Spa66); SEQ ID NO:48 (33 amino acids long and designated Spa67); SEQ ID NO:49 (36 amino acids long and designated Spa68); SEQ ID NO:50 (35 amino acids long and designated Spa69); SEQ ID NO:51 (34 amino acids long and designated Spa70); SEQ ID NO:52 (33 amino acids long and designated Spa71); SEQ ID NO:53 (32 amino acids long and designated Spa72); SEQ ID NO:54 (34 amino acids long and designated Spa73); SEQ ID NO:55 (33 amino acids long and designated Spa74); SEQ ID NO:56 (32 amino acids long and designated Spa75); SEQ ID NO:57 (31 amino acids long and designated Spa76); SEQ ID NO: 58 (30 amino acids long) designated Spa77); SEQ ID NO:59 (33 amino acids long and designated Spa78); SEQ ID NO:60 (32 amino acids long and designated Spa79); SEQ ID NO:61 (31 amino acids long and designated Spa80); SEQ ID NO: 62 (30 amino acids long and designated Spa81); SEQ ID NO:63 (29 amino acids long and designated Spa82); SEQ ID No:64 (32 amino acids long and designated Spa83); SEQ ID NO:65 (31 amino acids long and designated Spa84); SEQ ID NO:66 (30 amino acids long) designated Spa85); SEQ ID NO: 67 (29 amino acids long and designated Spa86); and SEQ ID NO:68 (28 amino acids long and designated Spa87).

21. The fragment of claim 13, wherein the fragment is fused to a second polypeptide.

22. A composition of any of claims 12, 13, or 19, further comprising a pharmaceutically acceptable carrier.

23. The composition of claim 22, further comprising an anti-cancer agent.

24. The composition of claim 22, wherein the composition is in a sustained release formulation.

25. A polypeptide comprising a fragment of the full-length pRB2/p130 spacer domain (616-828) or a variant of the fragment, wherein the fragment or the variant is between 11 and 38 amino acids long, wherein the polypeptide is capable of inhibiting cdk2 kinase activity, and wherein the fragment comprises SEQ ID NO:69 (11 amino acids long and designated Spa53).

26. A polypeptide consisting essentially of a fragment of the full-length pRB2/p130 spacer domain or a variant of the fragment, wherein the fragment or the variant is between 11 and 38 amino acids long, wherein the polypeptide is capable of inhibiting cdk2 kinase activity, and wherein the fragment comprises SEQ ID NO:69 (11 amino acids long and designated Spa53).

27. A method of treating cancer in a patient, comprising:
administering to the patient a therapeutically effective amount of a polypeptide comprising a fragment of the full-length pRB2/p130 spacer domain (616-828) or a variant of the fragment, wherein the fragment or the variant is between 9 and 38 amino acids long, wherein the fragment has at least 9 contiguous amino acids of Spa310 (641-679), and wherein the polypeptide is capable of inhibiting cdk2 kinase activity.

28. A method of treating cancer in a patient, comprising:
administering to the patient a therapeutically effective amount of a polypeptide consisting essentially of a fragment of the full-length pRB2/p130 spacer domain (616-828) or a variant of the fragment, wherein the fragment or the variant is between 9 and 38 amino acids long, wherein the fragment has at least 9 contiguous amino acids of Spa310 (641-679), and wherein the fragment is capable of inhibiting cdk2 kinase activity.

29. The method of claim 27 or 28, wherein the fragment is selected from the group consisting of SEQ ID NO:22 (9 amino acids long and designated Spa40); SEQ ID NO:23 (11 amino acids long and designated Spa41); SEQ ID NO:24 (14 amino acids long and designated Spa42); SEQ ID NO:25 (25 amino acids long and designated Spa43); SEQ ID NO:26 (30 amino acids long and designated Spa44); SEQ ID NO:27 (36 amino acids long and designated Spa45); SEQ ID NO:28 (14 amino acids long and designated Spa46); SEQ ID NO:29 (19 amino acids long and designated Spa47); SEQ ID NO:30 (24 amino acids long and designated Spa48); SEQ ID NO: 31 (29 amino acids long and designated Spa49; SEQ ID NO:32 (34 amino acids long and designated Spa50); SEQ ID NO:33 (20 amino acids long and designated Spa51); SEQ ID NO:34 (9 amino acids long and designated Spa52); SEQ ID NO:39 (38 amino acids long and designated Spa58); SEQ ID NO:40 (37 amino acids long and designated Spa59);
SEQ ID NO:41 (35 amino acids long and designated Spa60); SEQ ID NO:42 (34 amino acids long and designated Spa61); SEQ ID NO:43 (33 amino acids long and designated Spa62); SEQ ID NO:44 (37 amino acids long and designated Spa63); SEQ ID NO:45 (36 amino acids long and designated Spa64); SEQ ID NO:46 (35 amino acids long and designated Spa65); SEQ ID NO:47 (34 amino acids long and designated Spa66); SEQ ID NO:48 (33 amino acids long and designated Spa67); SEQ ID NO:49 (36 amino acids long and designated Spa68); SEQ ID NO:50 (35 amino acids long and designated Spa69); SEQ ID NO:51 (34 amino acids long and designated Spa70); SEQ ID NO:52 (33 amino acids long and designated Spa71); SEQ ID NO:53 (32 amino acids long and designated Spa72); SEQ ID NO:54 (34 amino acids long and designated Spa73); SEQ ID NO:55 (33 amino acids long and designated Spa74); SEQ ID NO:56 (32 amino acids long and designated Spa75); SEQ ID NO:57 (31 amino acids long and designated Spa76); SEQ ID NO: 58 (30 amino acids long) designated Spa77); SEQ ID NO:59 (33 amino acids long and designated Spa78); SEQ ID NO:60 (32 amino acids long and designated Spa79); SEQ ID NO:61 (31 amino acids long and designated Spa80); SEQ ID NO: 62 (30 amino acids long and designated Spa81); SEQ ID NO:63 (29 amino acids long and designated Spa82); SEQ ID No:64 (32 amino acids long and designated Spa83); SEQ ID NO:65 (31 amino acids long and designated Spa84); SEQ ID NO:66 (30 amino acids long) designated Spa85); SEQ ID NO: 67 (29 amino acids long and designated Spa86); and SEQ ID NO:68 (28 amino acids long and designated Spa87).

30. A method of treating cancer in a patient, comprising:
administering to the patient a therapeutically effective amount of a polypeptide comprising a fragment of the full-length pRB2 μl 30 spacer domain (616-828) or a variant of the fragment, wherein the fragment is selected from the group consisting of SEQ ID NO:35 (39 amino acids long and designated Spa54), SEQ ID NO:36 (39 amino acids long and designated Spa55); SEQ ID NO:37 (39 amino acids long and designated Spa56); and SEQ ID NO:38 (39 amino acids long and designated Spa57), and wherein the polypeptide is capable of inhibiting cdk2 kinase activity.

31. A method of treating cancer in a patient, comprising:
administering to the patient a therapeutically effective amount of a polypeptide consisting essentially of a fragment of the full-length pRB2/p130 spacer domain (616-828) or a variant of the fragment, wherein the fragment is selected from the group consisting of SEQ ID NO:35 (39 amino acids long and designated Spa54), SEQ ID NO: 36 (39 amino acids long and designated Spa55); SEQ ID NO:37 (39 amino acids long and designated Spa56); and SEQ ID NO:38 (39 amino acids long and designated Spa57), and wherein the fragment is capable of inhibiting cdk2 kinase activity.

32. A method of treating cancer in a patient, comprising:
administering to the patient a therapeutically effective amount of a polypeptide comprising a fragment of the full-length pRB2/p130 spacer domain or a variant of the fragment, wherein the fragment or the variant is between 11 and 38 amino acids long, wherein the polypeptide is capable of inhibiting cdk2 kinase activity, wherein the fragment comprises SEQ ID NO:69 (11 amino acids long and designated Spa53).

33. A method of treating cancer in a patient, comprising:
administering to the patient a therapeutically effective amount of a polypeptide consisting essentially of a fragment of the full-length pRB2/p130 spacer domain or a variant of the fragment, wherein the fragment or the variant is between 11 and 38 amino acids long, wherein the polypeptide is capable of inhibiting cdk2 kinase activity, and wherein the fragment comprises SEQ ID NO:69 (11 amino acids long and designated Spa53).
